(19) United States
(12) Patent Application Publication
(10) Pub. No.: US 2026/0114737 A1
(43) Pub. Date: Apr. 30, 2026

(54) Using Physiological Sensor Data From Biomedical Sensors For Automated Task Status Tracking And Management

(71) Applicant: Oracle International Corporation, Redwood Shores, CA (US)

(72) Inventor: Jennifer Darmour, Seattle, WA (US)

(73) Assignee: Oracle International Corporation, Redwood Shores, CA (US)

(21) Appl. No.: 19/371,422

(22) Filed: Oct. 28, 2025

Related U.S. Application Data
(60) Provisional application No. 63/713,460, filed on Oct. 29, 2024.

(51) Int. Cl. A61B 5/0205; A61B 5/00

(52) U.S. Cl. CPC A61B 5/0205; A61B 5/4833; A61B 5/7267; A61B 5/7435

(57) ABSTRACT

Techniques digitally tracking completion of user tasks based on physiological data obtained from biomedical sensors are disclosed. One or more embodiments manage a schedule of tasks for a specific user and presents the schedule to the user or to other users that have authorization to access the schedule (e.g., an authorized health care professional). In some embodiments, a system dynamically tracks the status of the tasks by obtaining physiological data from biomedical sensors of health monitoring devices. The physiological data may indicate if a particular task has been completed, as well as details related to the task. The system updates a record corresponding to the task based on the physiological data, thereby enabling the system to dynamically provide timely up-to-date tracking details regarding the schedule of tasks to the user.

### BENEFIT CLAIMS; RELATED APPLICATIONS; INCORPORATION BY REFERENCE

This application claims the benefit of U.S. Provisional Patent Application 63/713,460, filed Oct. 29, 2024, that is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to digitally tracking completion of user tasks. In particular, the present disclosure relates to digitally tracking completion of user tasks based on data obtained from external data sources, such as sensors of health monitoring devices and third-party applications

### BACKGROUND

Digital healthcare includes a growing field of technology that digitally transforms multiple aspects of healthcare. Some common examples include, but are not limited to, text-based conversational artificial intelligence (AI), mobile health applications, electronic health records (EHRs), and telehealth interfaces. Digital healthcare facilitates digital transformation in healthcare by incorporating software, hardware, and networking into healthcare delivery systems.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for using physiological data from biomedical sensors for automated task status tracking and management in accordance with one or more embodiments;

FIG. 3A illustrates a user interface displaying a schedule of tasks in accordance with one or more embodiments;

FIG. 3B illustrates a user interface displaying the schedule of tasks subsequent to status data for a subset of the tasks being updated based on physiological data obtained from biomedical sensors in accordance with one or more embodiments;

FIG. 4 illustrates a machine learning (ML) engine in accordance with one or more embodiments;

FIG. 5 illustrates an example set of operations of an ML engine in accordance with one or more embodiments; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. General Overview
2. Task Management Architecture
3. Managing Task Status Based On Sensor Data From Biomedical Sensors
4. Example Embodiments
5. Ml Architecture
   6. Practical Applications, Advantages, And Improvements
7. Computer Networks And Cloud Networks
8. Hardware Overview
9. Miscellaneous; Extensions

### 1. General Overview

One or more embodiments digitally track and manage user tasks in real-time based on physiological data obtained from biomedical sensors of health monitoring devices. User tasks, as referred to herein, include tasks that are to be performed by users. Digitally tracking a user task includes determining a status of the user task and/or detecting completion of the user task. The physiological data obtained from biomedical sensors may itself indicate the status of a user task. Alternatively, or additionally, a system deduces the status of a user task based on information included in the physiological data received from biomedical sensors of health monitoring devices.

User tasks directed towards improving or maintaining a user's health may include obtaining a data reading (e.g., blood pressure, pulse, blood glucose level) from a health monitoring device, taking medication, or performing exercise. In an example, the system updates a user task of “take insulin” as completed based on physiological data received from a blood insulin monitor, that is reflective of the user taking insulin.

One or more embodiments manage a schedule of tasks for a specific user and present the schedule to the user or to other users that have authorization to access the schedule (e.g., an authorized health care professional). The system tracks the status of the tasks, included in the schedule, in real-time by obtaining physiological data from biomedical sensors of health monitoring devices (e.g., a glucose monitor, a blood pressure monitor, a pulse oximeter, a heart rate monitor, or an activity tracker). The physiological data may indicate if a particular task has been completed, as well as details related to the task. The degree of completion required to satisfy a threshold level of completeness for determining if a task has been completed may vary between different embodiments. For example, in some embodiments, the system may require that the user has fully completed the task to determine that the task has been completed, whereas, in other embodiments, the system may only require that the user has initiated the task or partially completed the task to determine that the task has been completed. In some embodiments, the system requires only that the user has initiated a particular task to determine that the user has completed the particular task, while requiring that the user has fully completed another particular task to determine that the user has completed the other particular task.

The system updates a record corresponding to the task based on the physiological data, thereby enabling the system to dynamically provide timely up-to-date tracking details regarding the schedule of tasks to the user. These features also enable the system to provide timely up-to-date tracking details regarding the schedule of tasks to downstream computer processes that consume these task-related details. The system organizes and ranks the tasks in the schedule based on a respective status of each task.

One or more embodiments use an ML algorithm to modify a record of a task based on physiological data obtained from one or more biomedical sensors of health monitoring devices. In one example, the system modifies the time for which a task is scheduled, as indicated in the corresponding record of the task record, based on the physiological data. For example, using the ML algorithm, the system may change the timing for the user to obtain a blood pressure measurement from the morning to the afternoon based on previously-obtained physiological data, such as previously-obtained blood pressure measurements. By using this ML algorithm, the system is able to automatically make intelligent adjustments to the schedule of tasks for the user.

One or more embodiments use an ML algorithm to add an additional task to the schedule of tasks based on physiological data obtained from one or more biomedical sensors of health monitoring devices. In one example, the system adds a record for an additional task to a set of records of tasks for the user based on the physiological data. For example, using the ML algorithm, the system may add a task for obtaining a blood glucose measurement to the schedule of tasks based on previously obtained physiological data. By using this ML algorithm, the system can automatically make intelligent adjustments to the schedule of tasks for the user.

One or more embodiments described in this Specification and/or recited in the claims may not be included in this General Overview section.

### 2. Task Management Architecture

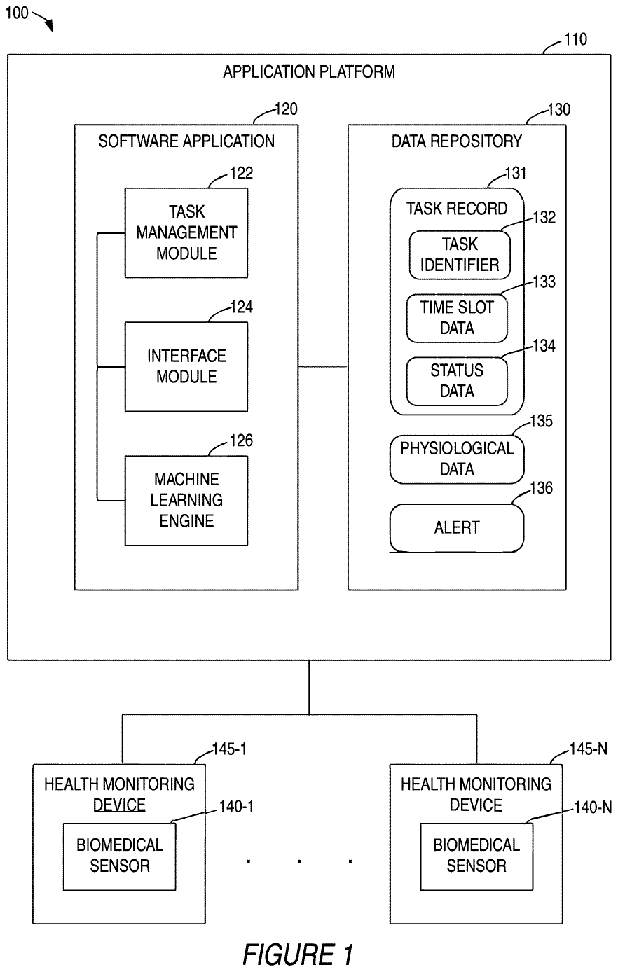

FIG. 1 illustrates a task management system 100 in accordance with one or more embodiments. As illustrated in FIG. 1, task management system 100 includes a software application 120 and a data repository 130 that are hosted within an application platform 110. In an embodiment, the software application 120 includes a task management module 122, an interface module 124, and an ML engine 126. In one or more embodiments, the task management system 100 may include more or fewer components than the components illustrated in FIG. 1. The components illustrated in FIG. 1 may be local to or remote from each other.

The components illustrated in FIG. 1 may be implemented in software and/or hardware. Each component may be distributed over multiple applications and/or machines. Multiple components may be combined into one application and/or machine. Operations described with respect to one component may instead be performed by another component.

The components of the system 100 may communicate with one another via one or more computer networks. Furthermore, one or more components of the system 100 may be implemented as part of a cloud network. Additional embodiments and/or examples relating to computer networks are described below in Section 7, titled “Computer Networks and Cloud Networks.”

In some embodiments, the application platform 110 includes a computing infrastructure on which software applications are hosted and executed. For example, the application platform 110 may include physical hardware devices and virtual resources, such as servers, storage, operating systems, and networks. The application platform 110 can be centralized in a data center or decentralized across multiple data centers.

In one or more embodiments, the software application 120 includes a digital healthcare application that provides intelligent tools for data-driven healthcare experiences. For example, the software application 120 may be configured to facilitate the management of tasks that are directed towards a user's health. Examples of such tasks include, but are not limited to, obtaining a data reading (e.g., blood pressure, pulse, blood glucose level) from a health monitoring device, taking medication, performing exercise, or scheduling an appointment with a health care provider. Other types of tasks are also within the scope of the present disclosure. Furthermore, the software application 120 may include other types of software applications other than a digital healthcare application.

In one or more embodiments, data repository 130 is any type of storage unit and/or device (e.g., a file system, database, collection of tables, or any other storage mechanism) for storing data. Further, data repository 130 may include multiple different storage units and/or devices. The multiple different storage units and/or devices may or may not be of the same type or located at the same physical site. Further, data repository 130 may be implemented or executed on the same computing system as software application 120. Additionally, or alternatively, a data repository 130 may be implemented or executed on a computing system separate from software application 120. The data repository 130 may be communicatively coupled to software application 120 via a direct connection or via a network. Furthermore, data sets illustrated within the data repository 130 may include may be implemented across any of components within the task management system 100, in a decentralized and/or distributed manner. The data sets are illustrated within the data repository 130 for purposes of clarity and explanation.

In one or more embodiments, the task management module 122 is a software component configured to store, in the data repository 130 of the application platform 110, a set of task records 131 corresponding to a set of tasks to be completed by a user of the software application 120 hosted on the application platform 110. In some embodiments, the set of task records 131 includes (a) corresponding identifiers 132 for the tasks, (b) corresponding time slot data 133 for the tasks, and (c) corresponding status data 134 for the tasks.

The identifier 132 for a task includes any sequence of characters that uniquely identifies the task. In some embodiments, the identifier 132 includes a natural language label or instruction for the task. For example, the identifier 132 for a task may read “register your blood glucose levels.” Other types of identifiers 132 are also within the scope of the present disclosure.

The time slot data 133 indicates a corresponding time slot for which the corresponding task is to be completed. In one or more embodiments, the time slot includes a specific point in time (e.g., 9:00 am), a window of time (e.g., 9:00 am-11:00 am), or a natural language label for a period of time (e.g., morning). Other types of time slots and time slot data 133 are also within the scope of the present disclosure.

The status data 134 indicates if the corresponding task has been completed. In some embodiments, the status data 134 includes a flag value that indicates if the corresponding task has been completed. For example, the status data 134 may include a Boolean indicator such as “true” or “false.” Additionally, the status data 134 may also include details of a completed task. In one example in which a task for registering a blood pressure measurement has been completed by the user, the status data 134 for the task includes the specific blood pressure measurement obtained by the user in addition to a flag value set to “true.”

In one or more embodiments, the data repository 130 is any type of storage unit and/or device (e.g., a file system, database, collection of tables, or any other storage mechanism) for storing data. Further, the data repository 130 may include multiple different storage units and/or devices. The multiple different storage units and/or devices may or may not be of the same type or located at the same physical site.

In one or more embodiments, interface module 124 refers to hardware and/or software configured to facilitate communications between a user and software application 120. Interface module 124 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface module 124 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, interface module 124 is specified in one or more other languages, such as Java, C, or C++.

In some embodiments, the interface module 124 is configured to display tasks in a schedule within a user interface of the software application 120. The interface module 124 is also configured to obtain physiological data 135 from one or more biomedical sensors 140 (e.g., biomedical sensors 140-1 to 140-N, where N is an integer greater than 1) that are external to the application platform 110. In an embodiment, physiological data 135 is stored in data repository 130 where task management module 122, interface module 124, and ML engine 126 may access it for subsequent processing in executing their respective functions.

A biomedical sensor 140 is an electronic device that converts biological, chemical, or physical signals from the body into measurable electrical signals. Examples of biological signals include, but are not limited to, DNA, RNA, hormones, antigens, antibodies, enzymes, and microbe. Examples of chemical signals include, but are not limited to, concentration and ingredients of body liquids, such as glucose concentration and pH value. Examples of physical signals include, but are not limited to, blood pressure and body temperature. Other types of signals and health monitoring devices are also within the scope of the present disclosure.

Physiological data 135 includes measurements that capture the internal biological functions and physical states of the human body, such as heart rate, skin temperature, blood oxygen levels, electrodermal activity, and brainwave patterns. These data reflect how the body is functioning rather than what the body is doing. Physiological data 135 is distinct from activity data, as activity data describes external or behavioral aspects of movement and action (e.g., steps taken, distance traveled, posture, or gesture patterns) indicating what actions a person performs rather than their underlying physiological condition. In some embodiments, physiological data 135 includes hematological data, clinical biochemistry data, a glucose level of a user, a blood pressure of a user, an oxygen saturation level of a user, or a heart rate of a user. Other types of physiological data 135 are within the scope of the present disclosure. The physiological data 135 may indicate completion of one or more of the set of tasks of the task records 131.

In one or more embodiments, the biomedical sensors 140 are included in one or more health monitoring devices 145 (e.g., health monitoring devices 145-1 to 145-N, where N is an integer greater than 1). Health monitoring device 145 include tools or systems designed to continuously or periodically collect, measure, and record data related to an individual's physical or physiological state. These devices assess indicators such as heart rate, blood pressure, activity level, temperature, or other health-related metrics to support the detection, management, and prevention of medical conditions or to promote overall well-being. Health monitoring devices 145 may include, but are not limited to, wearable devices (e.g., smartwatches, fitness trackers, chest strap heart rate monitors, continuous glucose monitors, wearable electrocardiogram patches, and smart rings), home health monitoring devices (e.g., digital blood pressure monitors, pulse oximeters, digital thermometers, and smart inhalers), and clinical and remote patient monitoring devices (e.g., electrocardiogram monitors, ambulatory blood pressure monitors, wearable biosensors, and smart implants). Other types of health monitoring devices 145 are also within the scope of the present disclosure. Although the example illustrated in FIG. 1 shows a 1:1 ration between biomedical sensors 140 and health monitoring devices 145 (e.g., biomedical sensor 140-1 included in health monitoring device 145-1, . . . , biomedical sensor 140-N included in health monitoring device 145-N), in some embodiments, multiple biomedical sensors 140 are included in a single health monitoring device 145.

In some embodiments, interface module 124 is configured to obtain multiple instances of the same type of physiological data 135 from one or more biomedical sensors 140 over a period of time. For example, interface module 124 may be configured to obtain a single blood pressure measurement of a user at the same particular time (e.g., 11:00 AM) every day. An instance of physiological data 135 refers to a single, time-stamped (or otherwise specific to a particular point in time) measurement that reflects a specific aspect of an individual's biological or physiological state, as detected and recorded by a medical sensing device. This instance represents a discrete data point-such as a heart rate value, skin temperature reading, or blood oxygen saturation level-captured under defined conditions. An instance of physiological data 135 may be associated with sensor metadata (e.g., time, location, and/or device ID). In one example.

In one or more embodiments, task management module 122 is configured to determine if a user has completed a task based on an instance of physiological data 135 and the time slot data 133 of the task. For example, if the instance of physiological data 135 indicates that the task has been completed in accordance with the time slot indicated by the time slot data 133 of the task (e.g., on a particular day prior to a specific point in time or within a particular window of time), then the task management module 122 may determine that the user has completed the task. If the instance of physiological data 135 indicates that the task has not been completed in accordance with the time slot indicated, then the task management module 122 may determine that the user has not completed the task. In some embodiments, the task management module 122 is configured to, responsive to a determination that the user has completed the task, update the status data 134 of the task to indicate that the task has been completed. In some embodiments, the task management module 122 is also configured to, responsive to a determination that the user has not completed the task, generate an alert 136 to be displayed, by the interface module 124, on a computing device of the user. The alert 136 includes an identifier of the task to remind and prompt the user to complete the task. In an embodiment, the alert 136 is stored in data repository 130.

Data sources other than biomedical sensors 140 and health monitoring devices 145 may provide data that indicates if a user has completed a task. In some embodiments, one or more third-party applications (not shown) communicate such data (e.g., via a network connection) to the interface module 124 to be processed by the task management module 122 and the ML engine 126 using the same techniques as disclosed herein for the physiological data obtained from the biomedical sensors 140. For example, the task management module 122 may process such data from third-part applications to determine if a user has completed a task. The third-party applications may include any applications that are developed, deployed, and managed by entities (e.g., companies or organizations) other than the entity (e.g., company or organization) that developed, deployed, and manages the software application 120. The one or more third-party applications may include, but are not limited to, mobile applications, web applications, and messaging applications. Other types of third-party applications are also within the scope of the present disclosure.

In one or more embodiments, the interface module 124 is configured to obtain the data from a third-party application by displaying a notification on a computing device of the user via the third-party application. The notification may prompt the user to complete a task. For example, interface module 124 may transmit, to the user's smartphone, a text message that includes a reminder for the user to take a particular medication or for the user to take a blood pressure measurement. The notification may also prompt the user to respond to the notification with a confirmation that the task has been completed. For example, in the scenario mentioned above in which the text message includes a reminder for the user to take a particular medication, the text message may also include an instruction for the user to send a reply text message indicating that the user has taken the particular medication once the user has done so. In some embodiments, interface module 124 is configured to receive, from the third-party application, a response to the notification that includes an explicit confirmation that the user has completed the task. The response may also include specific details regarding the task. For example, if the notification prompts the user to register a blood pressure measurement, then the user may send a response that includes the blood pressure measurement once the user has taken the measurement.

The task management module 122 uses the task records 131 to map the physiological data 135 to specific health-related tasks, such as taking particular medications. In some embodiments, the task management module 122 uses the task records 131 and physiological data 135 to manage a medication list for a user. In one example, the task records 131 for the user include different tasks for taking different medications and the task management module 122 uses the physiological data 135 to track whether the user has taken the medications in the medication list in accordance with the corresponding time slot data 133. In some embodiments, the task management module 122 is configured to display, or otherwise provide, alerts 136 to the user when the task of taking a particular medication is missed.

In one or more embodiments, the ML engine 126 includes one or more ML algorithms that are configured to modify one or more of the task records 131 stored in the data repository 130 based at least in part on physiological data 135 obtained from one or more biomedical sensors 140. In an embodiment, the ML engine 126 is configured to modify the time for which a task is scheduled, as indicated by the time slot data 133 in the corresponding task record 131, based on the physiological data 135 obtained from one or more biomedical sensors 140. For example, the ML engine 126 may be configured to change the time for which the user is scheduled to take blood pressure medication from 6:00 pm to 1:00 pm based on physiological data 135 that includes blood pressure measurements, taken at 3:00 pm every day for the last three days, that indicate the user's blood pressure has been increasing above a predefined threshold value.

In some embodiments, the ML engine 126 is configured to train an ML model to modify one or more of the task records 131 as discussed above. In an embodiment, the ML engine 126 trains the ML model using training data that includes instances of historical physiological data 135, instances of identifiers 132 of historical tasks, and indications of times at which the historical tasks were completed. Depending on the nature of the task and physiological data, the ML engine 126 may use different ML paradigms to train the ML model. For example, the ML engine 126 may apply supervised learning, using a regression or classification model (e.g., random forest, gradient boosting, or neural network) to predict the optimal time offset or best time window based on physiological states preceding successful task outcomes. Other techniques for training the ML model to modify task records 131 are also within the scope of the present disclosure.

In one or more embodiments, the ML engine 126 includes one or more ML algorithms that are configured to add an additional task record 131 to the set of task records 131 of a user stored in the data repository 130 based at least in part on physiological data 135 obtained from one or more biomedical sensors 140. For example, the ML engine 126 may be configured to add an additional task record 131 for taking insulin based on physiological data 135 that includes a blood glucose measurement that indicates the user's blood glucose level is above a predefined threshold value.

In some embodiments, the ML engine 126 is configured to train an ML model to add an additional task record 131 to the set of task records 131 stored in the data repository 130 based at least in part on the physiological data 135 obtained from the biomedical sensors 140. In an embodiment, the ML engine 126 trains the ML model using training data that includes instances of historical physiological data 135, instances of identifiers 132 of historical tasks, and indications of times at which the historical tasks were completed. Depending on the nature of the task and physiological data 135, the ML engine 126 may use different ML paradigms to train the ML model. For example, the ML engine 126 may apply supervised learning, using a regression or classification model (e.g., random forest, gradient boosting, or neural network) to train the ML model to add task records 131 to the set of task records 131. Other techniques for training the ML model to add task records 131 are also within the scope of the present disclosure.

One or more components of the ML engine 126 may be implemented in accordance with the embodiments and/or examples relating to ML engine 400 described below in Section 5, titled “Machine Learning Architecture.”

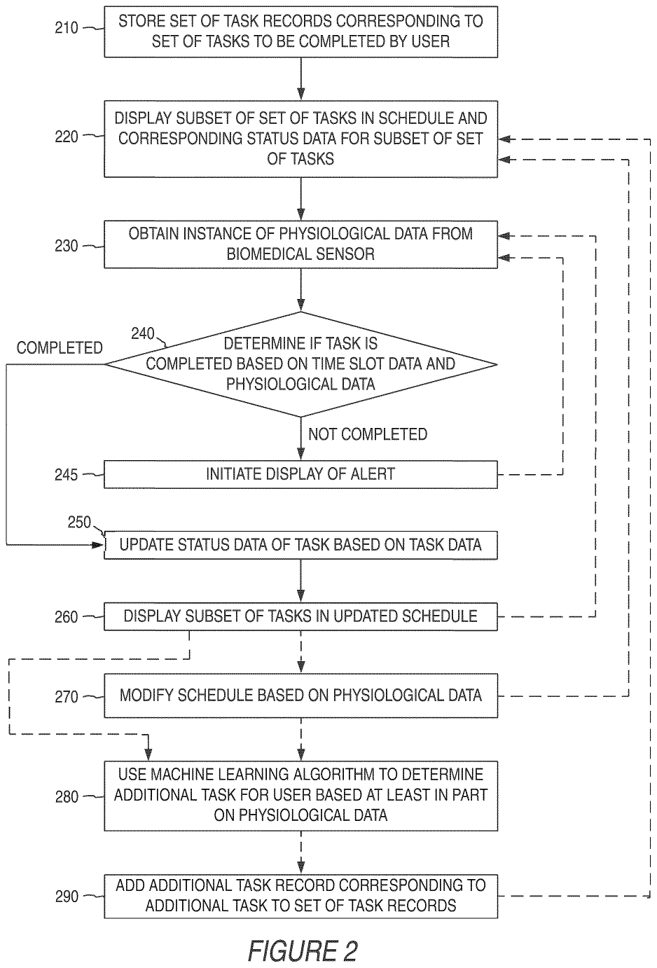

In one or more embodiments, task management system 100 refers to hardware and/or software configured to perform operations described herein for using physiological data from biomedical sensors for automated task status tracking and management. Examples of operations for using physiological data from biomedical sensors for automated task status tracking and management are described below with reference to FIG. 2.

In an embodiment, task management system 100 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

### 3. Managing Task Status Based On Sensor Data From Biomedical Sensors

FIG. 2 illustrates an example set of operations for using physiological data from biomedical sensors for automated task status tracking and management in accordance with one or more embodiments. One or more operations illustrated in FIG. 2 may be modified, rearranged, or omitted all together. Accordingly, the particular sequence of operations illustrated in FIG. 2 should not be construed as limiting the scope of one or more embodiments.

In an embodiment, the system stores, in a data repository of an application platform, a set of task records corresponding to a set of tasks to be completed by a user of a software application hosted on the application platform (Operation 210). The set of task records includes (a) corresponding identifiers for the respective tasks in the set of tasks, (b) corresponding time slot data for the respective tasks in the set of tasks, and (c) corresponding status data for the respective tasks in the set of tasks. In some embodiments, the user (or another user on behalf of the user, such as health care provider or professional) adds, edits, and removes task records in the data repository using the application platform. In one example, the task records stored in the data repository have been configured and added to the data repository via the application platform.

In one or more embodiments, the identifier for a task includes a natural language label or instruction for the task (e.g., “register your blood glucose levels”). However, other types of identifiers are also within the scope of the present disclosure. The time slot data indicates a corresponding time slot for which the corresponding task is to be completed (e.g., a specific point in time, a window of time, or a natural language label for a period of time). The status data indicates if the corresponding task has been completed. In some embodiments, the status data includes a flag value that indicates if the corresponding task has been completed. Additionally or alternatively, the status data includes details of a completed task. In one example in which a task for registering a blood pressure measurement has been completed by the user, the status data for the task includes the specific blood pressure measurement obtained by the user in addition to a flag value set to “true.”

In one or more embodiments, the system displays a subset of the set of tasks in a schedule within a user interface of the software application (Operation 220). The system orders the tasks in the schedule based on the corresponding time slot data for the tasks. For example, the system may cause the tasks to be displayed from top to bottom within the user interface in order of the earliest or soonest scheduled task to the latest scheduled task. The system also causes the corresponding status data for the tasks to be displayed in association with the tasks. For example, the system may display a particular graphic user interface element representing the completion status in association with (e.g., displayed adjacent to) the corresponding task in the displayed schedule. Examples of such graphic user interface elements include, but are not limited to, a checkmark to indicate that the corresponding task has been completed and an icon representing the corresponding task to indicate that the corresponding task has not yet been completed. Other types of graphic user interface elements are also within the scope of the present disclosure. Furthermore, if a task has been completed, then the system may also display one or more details of the completed task in association with the task. For example, if the user has completed a task of obtaining a health-related measurement (e.g., blood pressure measurement) using a health monitoring device (e.g., blood pressure monitor), then the system 100 may display the health-related measurement in association with the task in the schedule.

In some embodiments, the system obtains an instance of physiological data from a biomedical sensor of a health monitoring device (Operation 230). The system may obtain instances of physiological data in a variety of ways. In one or more embodiments, the system communicates with the health monitoring device via a network connection to obtain the physiological data collected by the biomedical sensor. In one example, the system periodically transmits, to the health monitoring device, a request for physiological data that was obtained by the biomedical sensor(s) of the health monitoring device subsequent to the last time the system transmitted such a request or the last time the health monitoring device sent physiological data to the system. In another example, the health monitoring device transmits physiological data to the system in response to the biomedical sensor collecting the physiological data.

In one or more embodiments, the physiological data includes, but is not limited to, hematological data, clinical biochemistry data, a glucose level obtained from a glucometer, a blood pressure level obtained from a blood pressure monitor, a pulse rate and oxygen saturation level obtained from a pulse oximeter, or a heart rate obtained from a heart rate monitor. Other types of physiological data are also within the scope of the present disclosure.

The physiological data itself is an indication that a particular corresponding task has been completed. For example, if the physiological data obtained by the system includes a blood pressure level of the user, then the system may interpret the presence of a blood pressure level in the physiological data as an indication that a corresponding task of obtaining a blood pressure reading has been completed. The system may obtain the physiological data by communicating with the health monitoring device directly (e.g., in instances in which the health monitoring device has wireless communication capabilities) or via a computing device to which the health monitoring device may connect (e.g., in instances in which the health monitoring device may connect to a computer that may connect to the system).

In addition or as an alternative to obtaining physiological data from biomedical sensors of health monitoring devices, the system may also obtain, from data sources other than biomedical sensors of health monitoring device, data that indicates if the user has completed a task in the set of tasks. In one or more embodiments, the system obtains such data by displaying a notification on a computing device of the user via a third-party application. The notification is configured to prompt the user to complete a task. For example, the system may transmit, to the user's smartphone, a text message that includes a reminder for the user to take a particular medication or for the user to take a blood pressure measurement. The notification may also include a prompting for the user to respond to the notification with a confirmation that the task has been completed. For example, in the scenario mentioned above in which the text message includes a reminder for the user to take a particular medication, the text message may also include an instruction for the user to send a reply text message indicating that the user has taken the particular medication once the user has done so. In some embodiments, the system receives, from the third-party application, a response to the notification and the response includes the data indicating that the user has taken the particular medication. For example, the data may include an explicit confirmation that the user has completed the task. The data may also include specific details regarding the task. For example, if the notification prompts the user to register a blood pressure measurement, then the user may send a response that includes the blood pressure measurement once the user has taken the measurement.

In some embodiments, the system determines if a task is completed based on an instance of physiological sensor data obtained from the biomedical sensor and time slot data for the task (Operation 240). The system may determine if the instance of physiological data indicates that the task has been completed in accordance with the time slot indicated by the time slot data of the task. In one example, the system determines if the task has been completed on a particular day prior to a specific point in time (e.g., today, prior to 3:00 pm). In another example, the system determines if the task has been completed within a particular window of time (e.g., between 1:00 pm and 3:00 pm). In an embodiment, if the system determines that the instance of physiological data indicates that the task has been completed in accordance with the time slot indicated by the time slot data of the task, then the system determines that the user has completed the task. In an embodiment, if the system determines that the instance of physiological data indicates that the task has not been completed in accordance with the time slot, then the system determines that the user has not completed the task.

In one or more embodiments, the system, responsive to a determination that the user has not completed the task, displays an alert on a computing device of the user (Operation 245). For example, the system may generate an alert that includes an identifier of the task to remind and prompt the user to complete the task. The system may then transmit the generated alert to the computing device of the user for display on the computing device. In some embodiments, the alert is presented to the user via an e-mail that is sent to an e-mail account of the user, via a text message that is sent to a mobile device of the user, or a third-party mobile application on a mobile device of the user. Other ways of displaying the alert are also within the scope of the present disclosure.

Subsequent to displaying the alert on the computing device of the user, the system may return to obtaining another instance of physiological data from a biomedical sensor of a health monitoring device (Operation 230). The other instance of physiological data may be the same type of physiological data as the instance of physiological data obtained during the previous iteration of the operation. For example, both instances of physiological data may include a blood pressure measurement. The other instance of physiological data may also be a different type of physiological data as the instance of physiological data obtained during the previous iteration of the operation. For example, the previous instance of physiological data may include a blood pressure measurement and the other instance of physiological data may include a blood glucose measurement. Furthermore, the other instance of physiological data may be obtained from the same biomedical sensor or the same health monitoring device from which the instance of physiological data was obtained during the previous iteration of the operation. The other instance of physiological data may also be obtained from a different biomedical sensor or different health monitoring device from which the instance of physiological data was obtained during the previous iteration of the operation.

In an embodiment, the system, responsive to the determination that the task is completed, updates the first set of status data to indicate that the first task has been completed (Operation 250). For example, the system may update the status data by changing a flag value for the task from false to true, thereby indicating that the task has been completed. The system may also update the status data to include details of the completed task. For example, if the physiological data includes a blood pressure measurement, then, in addition to changing the flag value for the task from false to true, the system may also add the blood pressure measurement to the status data of the task.

In some embodiments, the physiological data obtained by the system from the biomedical sensor may itself indicate the status of a user task, which the system may then use as the basis for updating the status data of the task. For example, the physiological data may include sensor data that specifies a measurement (e.g., a blood pressure measurement) for a health-related measurement task. Alternatively, or additionally, the system may deduce the status of a task based on information that is included in the physiological data received from one or more biomedical sensors. For example, the system may deduce that the user has completed 30 minutes of cardiovascular exercise based on sensor data specifying that the user's heart rate was above a particular threshold level for a 30-minute period.

In one or more embodiments, the system displays the subset of the set of tasks in an updated schedule within the user interface of the software application (Operation 260). The system again orders the tasks in the schedule based on the corresponding time slot data for the tasks (e.g., causing the tasks to be displayed from top to bottom within the user interface in order of the earliest or soonest scheduled task to the latest scheduled task). The system may also display the corresponding status data for the tasks in association with the tasks. Furthermore, if a task has been completed, then the system may display one or more details of the completed task in association with the task. In an embodiment, the system displays the updated status data of the task (e.g., resulting from the obtained physiological data) in association with the task in the updated schedule. For example, if the schedule includes a task for registering a blood pressure measurement and recently-obtained physiological data includes the blood pressure measurement, then the system may display the recently-obtained blood pressure measurement in association with the task. Subsequent to displaying the subset of the set of tasks in an updated schedule within the user interface of the software application, the system may return to obtaining another instance of physiological data from a biomedical sensor of a health monitoring device (Operation 230).

Additionally or alternatively, the system may modify one or more aspects of the set of task records based at least in part on the instance(s) of physiological data. In some embodiments, the set of time slot data for the task includes a schedule for periodically performing the task and the system modifies the schedule based on one or more instances of physiological data (Operation 270). In one example, the system uses an ML algorithm to modify the timing for completing the task based at least in part on the physiological data. For example, if the system determines that user's blood pressure is high based on the physiological data, the system may move the task of taking blood pressure medication to an earlier time to help address the time-sensitivity of the situation. Subsequent to modifying the schedule, the system may return to displaying a subset of the set of tasks in a schedule within a user interface of the software application (Operation 220).

In an embodiment, subsequent to or instead of the system modifying the schedule, the system uses a machine learning algorithm to determine an additional task for the user based at least in part on one or more instances of physiological data (Operation 280). The additional task may include an additional instance of a task that is already included in the schedule of tasks. For example, based on physiological data that includes a blood pressure measurement that was taken in the morning in accordance with a corresponding task scheduled for the morning, the machine learning algorithm may determine that an additional blood pressure measurement should be taken in the afternoon (e.g., based on a determination that the earlier blood pressure measurement is above a particular threshold level). The additional task may also include a new task that is not yet included in the schedule of tasks. For example, based on physiological data that includes a blood pressure measurement in accordance with a corresponding task included in the schedule, the machine learning algorithm may determine that a heart rate measurement should be taken (e.g., based on a determination that the blood pressure measurement is below a particular threshold level).

In an embodiment, the system adds, to the set of task records stored in the data repository, an additional task record corresponding to the additional task (Operation 290). The system may automatically add the additional task record to the set of task records in response to the system determining, using the machine learning algorithm, the additional task for the user. Alternatively, the system may transmit a notification of the additional task determined by the machine learning algorithm to an authorized health care professional and obtain an instruction or authorization from the authorized health care professional prior to adding the additional task record to the set of task records. The system may then return to displaying a subset of the set of tasks in a schedule within the user interface of the software application (Operation 220).

### 4. Example Embodiments

A detailed example is described below for purposes of clarity. Components and/or operations described below should be understood as one specific example that may not be applicable to certain embodiments. Accordingly, components and/or operations described below should not be construed as limiting the scope of any of the claims.

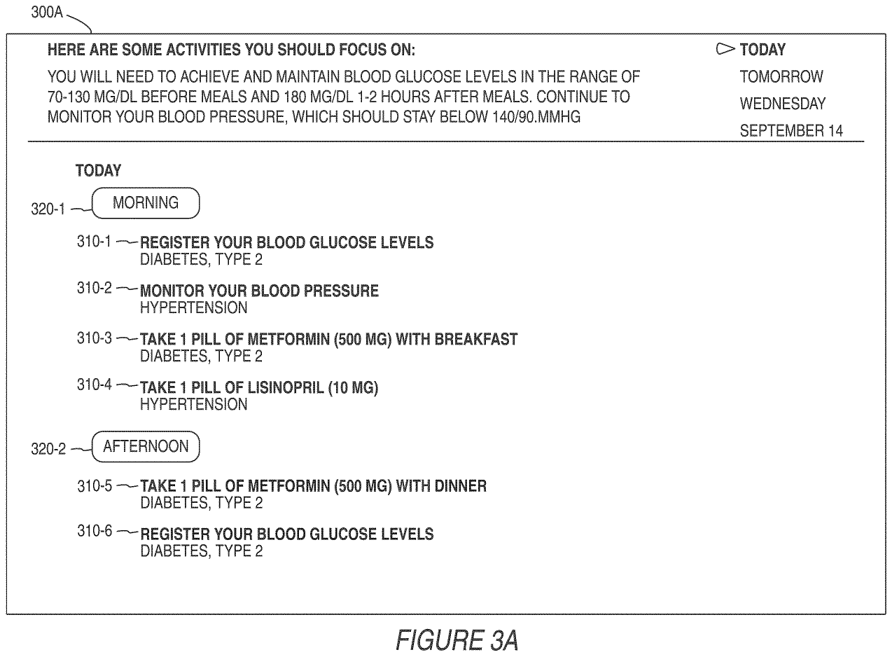

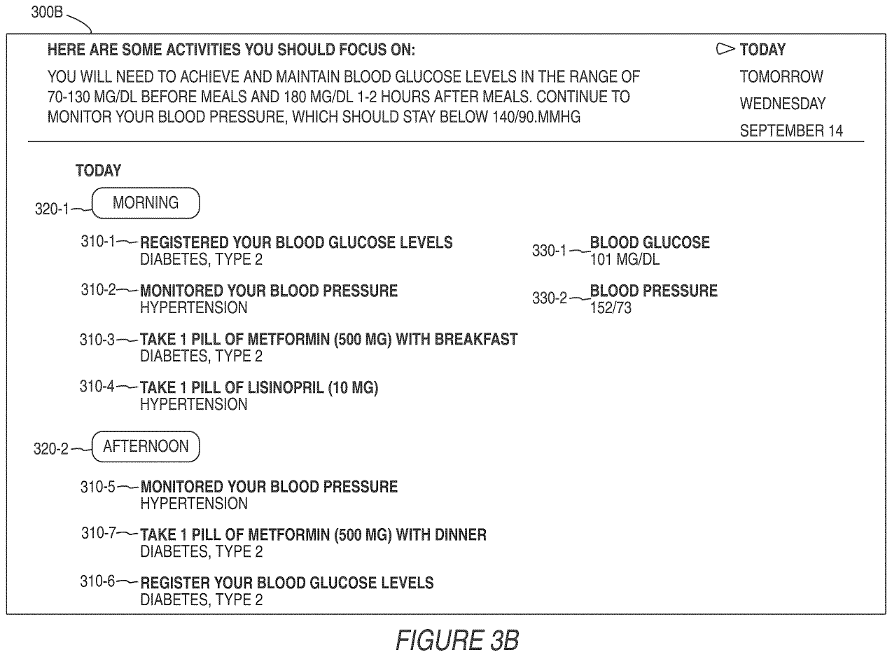

FIG. 3A illustrates a user interface 300A displaying a schedule of tasks 310 in accordance with one or more embodiments. In the example shown in FIG. 3A, the schedule of tasks 310 includes tasks 310-1, 310-2, 310-3, 310-4, 310-5, and 310-6 displayed in the schedule based on their respective time slots 320 indicated by their time slot data, from top to bottom within the user interface 300A in order of the earliest or soonest scheduled task 310-1 to the latest scheduled task 310-6. The time slots corresponding to the tasks 310 are displayed in association with their corresponding tasks 310. For example, tasks 310-1, 310-2, 310-3, and 310-4 are displayed in association with the “MORNING” time slot 320-1, while tasks 310-5 and 310-6 are displayed in association with the “AFTERNOON” time slot 320-2. In some embodiments, the user interface 300A displays indications of the respective statuses of the tasks 310 in association with the tasks 310. In the example shown in FIG. 3A, the indications include blank space to indicate that the tasks 310 are not yet completed. In other embodiments, an indication of a status of a task being not yet completed includes a text-based label (e.g., “Incomplete”) or a particular graphic element (e.g., an empty unchecked/unmarked box).

FIG. 3B illustrates a user interface 300B displaying the schedule of tasks 310 subsequent to status data for a subset of the tasks 310 being updated based on physiological data 330 obtained from biomedical sensors in accordance with one or more embodiments. In the example shown in FIG. 3B, the physiological data 330 includes a first instance of physiological data 330-1 and a second instance of physiological data 330-2. The first instance of physiological data 330-1 includes a blood glucose measurement of 101 mg/dL and the second instance of physiological data 330-2 includes a blood pressure measurement of 152/73. In FIG. 3B, the user interface 300B displays the first instance of physiological data 330-1 and the second instance of physiological data 330-2 as status data indicating that their respective tasks 310-1 and 310-2 have been completed. Additionally, in the example shown in FIG. 3B, the user interface 300B has included an additional task 310-7 of taking one pill of Metformin (500 mg) with dinner in order to treat the user for the risks of a high blood glucose level indicated by the first instance of physiological data 330-1.

### 5. Machine Learning Architecture

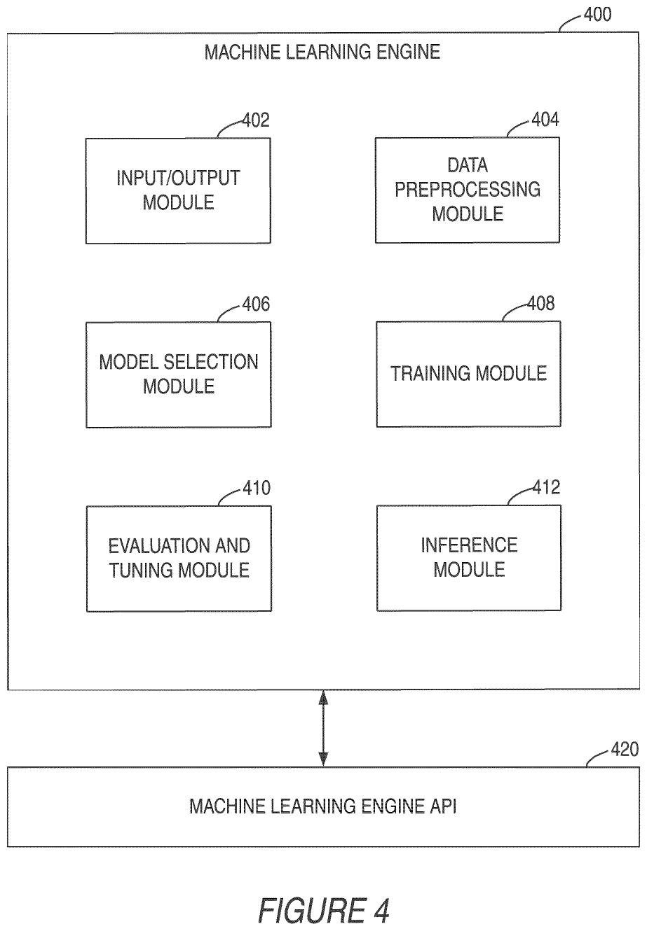

FIG. 4 illustrates a machine learning engine 400 in accordance with one or more embodiments. As illustrated in FIG. 4, machine learning engine 400 includes input/output module 402, data preprocessing module 404, model selection module 406, training module 408, evaluation and tuning module 410, and inference module 412.

In accordance with an embodiment, input/output module 402 serves as the primary interface for data entering and exiting the system, managing the flow and integrity of data. This module may accommodate a wide range of data sources and formats to facilitate integration and communication within the machine learning architecture.

In an embodiment, an input handler within input/output module 402 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 402 to be versatile in different operational contexts, whether processing historical datasets or streaming data.

In accordance with an embodiment, input/output module 402 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 402 includes an output framework designed to handle the distribution and exportation of outputs, predictions, or insights. Using the output framework, input/output module 402 formats these outputs into user-friendly and accessible formats, such as reports, visualizations, or data files compatible with other systems. Input/output module 402 also ensures secure and efficient transmission of these outputs to end-users or other systems in an embodiment and may employ encryption and secure data transfer protocols to maintain data confidentiality.

In accordance with an embodiment, data preprocessing module 404 transforms data into a format suitable for use by other modules in machine learning engine 400. For example, data preprocessing module 404 may transform raw data into a normalized or standardized format suitable for training ML models and for processing new data inputs for inference. In an embodiment, data preprocessing module 404 acts as a bridge between the raw data sources and the analytical capabilities of machine learning engine 400.

In an embodiment, data preprocessing module 404 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 404 may be configured to handle anomalies in different ways depending on context. Data preprocessing module 404 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 404 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 404 processes new data for inference, data preprocessing module 404 replicates the same preprocessing steps to ensure consistency with the training data format. This helps to avoid discrepancies between the training data format and the inference data format, thereby reducing the likelihood of inaccurate or invalid model predictions.

In an embodiment, model selection module 406 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 406 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 406 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 406 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 406 also considers computational efficiency and resource constraints. This is meant to help ensure the selected model is both accurate and practical in terms of computational and time requirements. In an embodiment, certain features of model selection module 406 are configurable such as a configured bias toward (or against) computational efficiency.

In accordance with an embodiment, training module 408 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 408 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 408 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 408 includes logic to handle different types of data and learning tasks. For instance, it includes different training routines for supervised learning (where the training data comes with labels) and unsupervised learning (without labeled data). In the case of deep learning models, training module 408 also manages the complexities of training neural networks that include initializing network weights, choosing activation functions, and setting up neural network layers.

In an embodiment, evaluation and tuning module 410 incorporates dynamic feedback mechanisms and facilitates continuous model evolution to help ensure the system's relevance and accuracy as the data landscape changes. Evaluation and tuning module 410 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 410 performs continuous model tuning by using hyperparameter optimization. Evaluation and tuning module 410 performs an exploration of the hyperparameter space using algorithms, such as grid search, random search, or more sophisticated methods like Bayesian optimization. Evaluation and tuning module 410 uses these algorithms to iteratively adjust and refine the model's hyperparameters—settings that govern the model's learning process but are not directly learned from the data—to enhance the model's performance. This tuning process helps to balance the model's complexity with its ability to generalize and attempts to avoid the pitfalls of underfitting or overfitting.

In an embodiment, evaluation and tuning module 410 integrates data feedback and updates the model. Evaluation and tuning module 410 actively collects feedback from the model's real-world applications, an indicator of the model's performance in practical scenarios. Such feedback can come from various sources depending on the nature of the application. For example, in a user-centric application like a recommendation system, feedback might include user interactions, preferences, and responses. In other contexts, such as predicting events, it might involve analyzing the model's prediction errors, misclassifications, or other performance metrics in live environments.

In an embodiment, feedback integration logic within evaluation and tuning module 410 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 410 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 412 transforms data raw data into actionable, precise, and contextually relevant predictions. In addition to processing and applying a trained model to new data, inference module 412 may also include post-processing logic that refines the raw outputs of the model into meaningful insights.

In an embodiment, inference module 412 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 412 transforms the outputs of a trained model into definitive classifications. Inference module 412 employs the underlying model as a tool to generate probabilistic outputs for each potential class. It then engages in an interpretative process to convert these probabilities into concrete class labels.

In an embodiment, when inference module 412 receives the probabilistic outputs from the model, it analyzes these probabilities to determine how they are distributed across some or every potential class. If the highest probability is not significantly greater than the others, inference module 412 may determine that there is ambiguity or interpret this as a lack of confidence displayed by the model.

In an embodiment, inference module 412 uses thresholding techniques for applications where making a definitive decision based on the highest probability might not suffice due to the critical nature of the decision. In such cases, inference module 412 assesses if the highest probability surpasses a certain confidence threshold that is predetermined based on the specific requirements of the application. If the probabilities do not meet this threshold, inference module 412 may flag the result as uncertain or defer the decision to a human expert.

Inference module 412 dynamically adjusts the decision thresholds based on the sensitivity and specificity requirements of the application, subject to calibration for balancing the trade-offs between false positives and false negatives.

In accordance with an embodiment, inference module 412 contextualizes the probability distribution against the backdrop of the specific application. This involves a comparative analysis, especially in instances where multiple classes have similar probability scores, to deduce the most plausible classification. In an embodiment, inference module 412 may incorporate additional decision-making rules or contextual information to guide this analysis, ensuring that the classification aligns with the practical and contextual nuances of the application.

In regression models, where the outputs are continuous values, inference module 412 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 412 incorporates domain-specific adjustments into its post-processing routine. This involves tailoring the model's output to align with specific industry knowledge or contextual information. For example, in financial forecasting, inference module 412 may adjust predictions based on current market trends, economic indicators, or recent significant events, ensuring that the outputs are both statistically accurate and practically relevant.

In an embodiment, inference module 412 includes logic to handle uncertainty and ambiguity in the model's predictions. In cases where inference module 412 outputs a measure of uncertainty, such as in Bayesian inference models, inference module 412 interprets these uncertainty measures by converting probabilistic distributions or confidence intervals into a format that can be easily understood and acted upon. This provides users with both a prediction and an insight into the confidence level of that prediction. In an embodiment, inference module 412 includes mechanisms for involving human oversight or integrating the instance into a feedback loop for subsequent analysis and model refinement.

In an embodiment, inference module 412 formats the final predictions for end-user consumption. Predictions are converted into visualizations, user-friendly reports, or interactive interfaces. In some systems, like recommendation engines, inference module 412 also integrates feedback mechanisms, where user responses to the predictions are used to continually refine and improve the model, creating a dynamic, self-improving system.

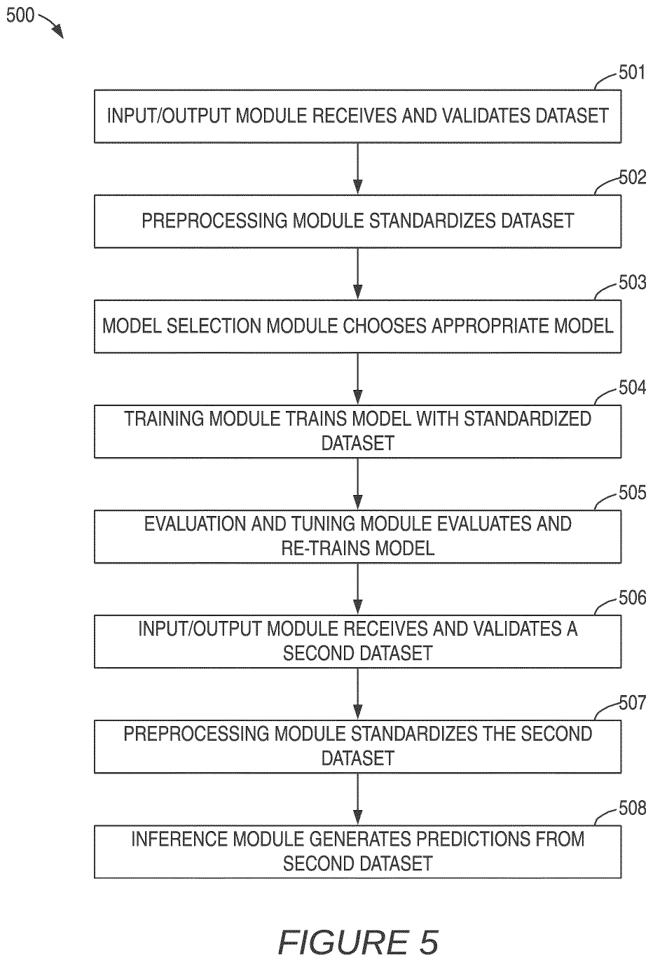

FIG. 5 illustrates the operation of a machine learning engine in one or more embodiments. In an embodiment, input/output module 402 receives a dataset intended for training (Operation 501). This data can originate from diverse sources, like databases or real-time data streams, and in varied formats, such as CSV, JSON, or XML. Input/output module 402 assesses and validates the data, ensuring its integrity by checking for consistency, data ranges, and types.

In an embodiment, training data is passed to data preprocessing module 404. Here, the data undergoes a series of transformations to standardize and clean it, making it suitable for training ML models (Operation 502). This involves normalizing numerical data, encoding categorical variables, and handling missing values through techniques like imputation.

In an embodiment, prepared data from the data preprocessing module 404 is then fed into model selection module 406 (Operation 503). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 408 trains the selected model with the prepared dataset (Operation 504). It implements learning algorithms to adjust the model's internal parameters, optimizing them to identify patterns and relationships in the training data. Training module 408 also addresses the challenge of overfitting by implementing techniques, like regularization and early stopping, ensuring the model's generalizability.

In an embodiment, evaluation and tuning module 410 evaluates the trained model's performance using the validation dataset (Operation 505). Evaluation and tuning module 410 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 402 receives a dataset intended for inference. Input/output module 402 assesses and validates the data (Operation 506).

In an embodiment, data preprocessing module 404 receives the validated dataset intended for inference (Operation 507). Data preprocessing module 404 ensures that the data format used in training is replicated for the new inference data, maintaining consistency and accuracy for the model's predictions.

In an embodiment, inference module 412 processes the new data set intended for inference, using the trained and tuned model (Operation 508). It applies the model to this data, generating raw probabilistic outputs for predictions. Inference module 412 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 420 allows for applications to leverage machine learning engine 400. In an embodiment, machine learning engine API 420 may be built on a RESTful architecture and offer stateless interactions over standard HTTP/HTTPS protocols. Machine learning engine API 420 may feature a variety of endpoints, each tailored to a specific function within machine learning engine 400. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 420 is equipped to support SOAP-based interactions. This extension involves defining a WSDL (Web Services Description Language) document that outlines the API's operations and the structure of request and response messages. In an embodiment, machine learning engine API 420 supports various data formats and communication styles. In an embodiment, machine learning engine API 420 endpoints may handle requests in JSON format or any other suitable format. For example, machine learning engine API 420 may process XML, and it may also be engineered to handle more compact and efficient data formats, such as Protocol Buffers or Avro, for use in bandwidth-limited scenarios.

In an embodiment, machine learning engine API 420 is designed to integrate WebSocket technology for applications necessitating real-time data processing and immediate feedback. This integration enables a continuous, bi-directional communication channel for a dynamic and interactive data exchange between the application and machine learning engine 400.

A generative model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component includes two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 402, when used for large language models, handles textual data, converting input text into a format that the model can process. This typically involves tokenization, where the text is broken down into manageable pieces, such as words or subwords, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 404 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 406, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 408, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 410 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 412, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 6. Practical Applications, Advantages, And Improvements

Embodiments provide several practical applications, advantages, and improvements over existing digital healthcare management solutions. One or more embodiments improve how application platforms and biomedical sensors function in digital healthcare management solutions by using physiological data obtained from biomedical sensors to dynamically track, in real-time, whether a user has completed a prescribed health-related task, such as taking insulin or blood pressure medication, within a defined time period, and to provide real-time alerts when non-completion is detected.

One or more embodiments improves the functioning of a computer system by improving the accuracy of digital healthcare and task management solutions. Instead of relying on user input or manual reporting, one or more embodiments automatically capture physiological data from biomedical sensors in real-time and send the physiological data to a task management system, thereby improving the task management system's ability to analyze the physiological data in real time to infer task completion. By immediately and dynamically providing the most recent physiological data to the task management system, one or more embodiments improve the accuracy of the determinations made by the task management system. Through real-time integration of multi-sensor inputs and automated determination, one or more embodiments reduce incorrect determinations based on out-of-date information compared to conventional reminder or manual-tracking systems. One or more embodiments thereby enhance the accuracy, speed, and reliability of computer-based health compliance monitoring.

One or more embodiments improve accuracy by relying on objective, sensor-derived physiological signals (e.g., glucose levels, heart rate variability) instead of subjective self-reporting or manual user input, and by using dynamic real-time algorithms to correlate those signals with task completion events. One or more embodiments also improve efficiency by automating the compliance-tracking process, eliminating the need for user intervention, and providing instantaneous, data-driven alerts that enable timely corrective actions. These technological features produce a real-world, tangible improvement in both the accuracy and responsiveness of health compliance systems.

One or more embodiments continuously monitor and adapts a schedule of tasks based on physiological trends or deviations, thereby providing a technological improvement in data-driven health monitoring, using adaptive algorithms and continuous sensor feedback rather than fixed schedules. For example, one or more embodiments automatically modifies a schedule for periodically performing a task based on physiological data obtained from a biomedical sensor (e.g., moving the task of taking blood pressure medication to an earlier time based on a user's blood pressure being high to help address the time-sensitivity of the situation). Additionally, one or more embodiments automatically adds tasks to a schedule of tasks for a user based on the physiological data (e.g., adding a task for taking insulin based on the user's glucose level being above a predefined threshold).

One or more embodiments provide an improved user interface for digital healthcare management solutions by automatically providing physiological data from biomedical sensors to a task management system on an application platform. For example, in order to update the status of a task being managed for the user by the task management system, the user only has to perform the task. There is no need for the user to log in and edit the status of the task manually. As a result, one or more embodiments reduce the number of steps involved in managing a schedule of tasks and task compliance, thereby improving the efficiency of user interaction with the task management system.

One or more embodiments can be applied to operations performed by one or more of the following: Database Software, Cloud Infrastructure Software, Customer Relationship Management Software, Data Science Software, Digital Assistant Software, Vision Software, Language Software, Speech Software, Forecasting Software, Enterprise Software, Middleware, Server Software, Identity Management Software, Application Development Software, Analytics Software, Security Software, Data Integration Software, Health Software, Hospitality Software, Retail Software, Utilities Software, Operating Systems, Virtualization Software, Governance and Administration Software, Migration & Disaster Recovery Software, Networking Software, Connectivity Software, Monitoring Software, Procurement Software, Project Management Software, Risk Management Software, Supply Chain Management Software, Manufacturing Software, Human Capital Management Software, Customer Experience Software, Advertising Software, and Industry-Specific Application Software.

### 7. Computer Networks And Cloud Networks

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis.

In an embodiment, a service provider provides a cloud network to one or more end users. Various service models may be implemented by the cloud network, including but not limited to Software-as-a-Service (SaaS), Platform-as-a-Service (PaaS), and Infrastructure-as-a-Service (IaaS). In SaaS, a service provider provides end users the capability to use the service provider's applications, that are executing on the network resources. In PaaS, the service provider provides end users the capability to deploy custom applications onto the network resources. The custom applications may be created using programming languages, libraries, services, and tools supported by the service provider. In IaaS, the service provider provides end users the capability to provision processing, storage, networks, and other fundamental computing resources provided by the network resources. Any arbitrary applications, including an operating system, may be deployed on the network resources.

In an embodiment, various deployment models may be implemented by a computer network, including but not limited to a private cloud, a public cloud, and a hybrid cloud. In a private cloud, network resources are provisioned for exclusive use by a particular group of one or more entities (the term “entity” as used herein refers to a corporation, organization, person, or other entity). The network resources may be local to and/or remote from the premises of the particular group of entities. In a public cloud, cloud resources are provisioned for multiple entities that are independent from each other (also referred to as “tenants” or “customers”). The computer network and the network resources thereof are accessed by clients corresponding to different tenants. Such a computer network may be referred to as a “multi-tenant computer network.” Several tenants may use a same particular network resource at different times and/or at the same time. The network resources may be local to and/or remote from the premises of the tenants. In a hybrid cloud, a computer network includes a private cloud and a public cloud. An interface between the private cloud and the public cloud allows for data and application portability. Data stored at the private cloud and data stored at the public cloud may be exchanged through the interface. Applications implemented at the private cloud and applications implemented at the public cloud may have dependencies on each other. A call from an application at the private cloud to an application at the public cloud (and vice versa) may be executed through the interface.

In an embodiment, tenants of a multi-tenant computer network are independent of each other. For example, a business or operation of one tenant may be separate from a business or operation of another tenant. Different tenants may demand different network requirements for the computer network. Examples of network requirements include processing speed, amount of data storage, security requirements, performance requirements, throughput requirements, latency requirements, resiliency requirements, Quality of Service (QoS) requirements, tenant isolation, and/or consistency. The same computer network may need to implement different network requirements demanded by different tenants.

In one or more embodiments, in a multi-tenant computer network, tenant isolation is implemented to ensure that the applications and/or data of different tenants are not shared with each other. Various tenant isolation approaches may be used.

In an embodiment, each tenant is associated with a tenant ID. Each network resource of the multi-tenant computer network is tagged with a tenant ID. A tenant is permitted access to a particular network resource only if the tenant and the particular network resources are associated with a same tenant ID.

In an embodiment, each tenant is associated with a tenant ID. Each application, implemented by the computer network, is tagged with a tenant ID. Additionally, or alternatively, each data structure and/or dataset, stored by the computer network, is tagged with a tenant ID. A tenant is permitted access to a particular application, data structure, and/or dataset only if the tenant and the particular application, data structure, and/or dataset are associated with a same tenant ID.

As an example, each database implemented by a multi-tenant computer network may be tagged with a tenant ID. Only a tenant associated with the corresponding tenant ID may access data of a particular database. As another example, each entry in a database implemented by a multi-tenant computer network may be tagged with a tenant ID. Only a tenant associated with the corresponding tenant ID may access data of a particular entry. However, the database may be shared by multiple tenants.

In an embodiment, a subscription list indicates application(s) that a tenant(s) is authorized to access. For each application, a list of tenant IDs of tenants authorized to access the application is stored. A tenant is permitted access to a particular application only if the tenant ID of the tenant is included in the subscription list corresponding to the particular application.

In an embodiment, network resources (such as digital devices, virtual machines, application instances, and threads) corresponding to different tenants are isolated to tenant-specific overlay networks maintained by the multi-tenant computer network. As an example, packets from any source device in a tenant overlay network may only be transmitted to other devices within the same tenant overlay network. Encapsulation tunnels are used to prohibit any transmissions from a source device on a tenant overlay network to devices in other tenant overlay networks. Specifically, the packets, received from the source device, are encapsulated within an outer packet. The outer packet is transmitted from a first encapsulation tunnel endpoint (in communication with the source device in the tenant overlay network) to a second encapsulation tunnel endpoint (in communication with the destination device in the tenant overlay network). The second encapsulation tunnel endpoint decapsulates the outer packet to obtain the original packet transmitted by the source device. The original packet is transmitted from the second encapsulation tunnel endpoint to the destination device in the same particular overlay network.

### 8. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

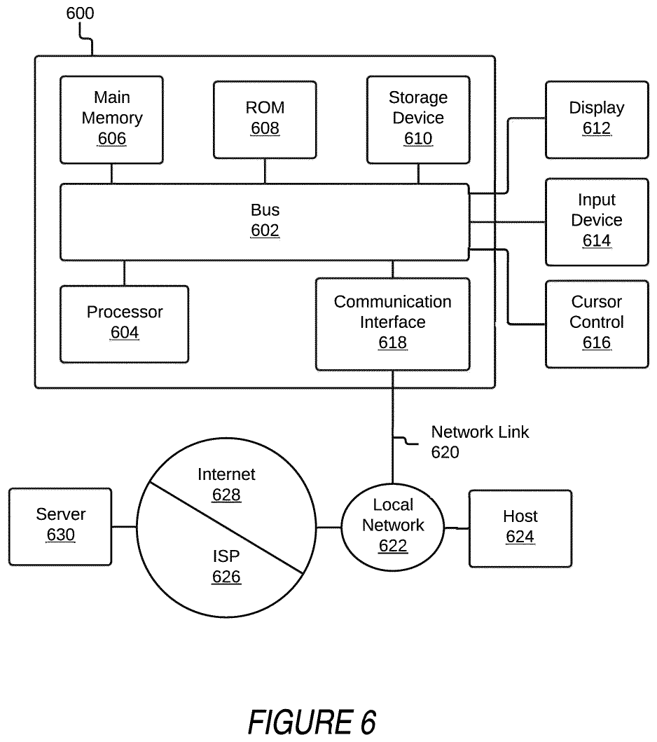

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may include non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that include bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 9. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media includes instructions which, when executed by one or more hardware processors, cause performance of any of the operations described herein and/or recited in any of the claims.

In an embodiment, a method includes operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer-readable media storing program instructions that, when executed by one or more hardware processors, cause performance of operations comprising:
storing, in a data repository of an application platform, a set of task records corresponding to a set of tasks to be completed by a user of a software application hosted on the application platform, a first task record of the set of task records comprising (a) a first set of time slot data for a first task indicating when the first task is to be completed, and (b) a first set of status data for the first task indicating that the first task has not been completed;
displaying a subset of tasks, of the set of tasks, in a schedule within a user interface of the software application, the subset of tasks being ordered in the schedule based on time slot data corresponding respectively to the subset of tasks;
displaying status data for the subset of tasks, the status data comprising the first set of status data indicating that the first task has not been completed;
obtaining a first instance of physiological data of the user from a biomedical sensor of a health monitoring device,
determining that the first task is completed based on the first instance of physiological data obtained from the biomedical sensor;
responsive to the determination that the first task is completed, updating the first set of status data to indicate that the first task has been completed; and
subsequent to updating the first set of status data, concurrently displaying within the user interface of the software application: (a) the subset of tasks including the first task and (b) the first set of status data indicating that the first task has been completed.

2. The non-transitory computer-readable media of claim 1, wherein:
a second task record of the set of task records comprises (a) a second set of time slot data for a second task indicating when the second task is to be completed, and (b) a second set of status data for the second task indicating that the second task has not been completed; and
the operations further comprise:
obtaining a second instance of physiological data of the user from the biomedical sensor of the health monitoring device, the second instance of physiological data indicating that the user has not completed the second task;
determining that the user has not completed the second task based on the time slot data corresponding to the second task and the second instance of physiological data; and
responsive to the determination that the user has not completed the second task, displaying an alert on a computing device of the user, the alert comprising an identifier of the second task.

3. The non-transitory computer-readable media of claim 2, wherein:
the second task comprises taking a particular medication;
wherein the second instance of physiological data indicates that the user has not taken the particular medication.

4. The non-transitory computer-readable media of claim 1, wherein:
the first task comprises taking a particular medication;
wherein taking the particular medication results in a physiological change for the user that is indicated by the first instance of physiological data.

5. The non-transitory computer-readable media of claim 1, wherein the health monitoring device comprises a glucose monitor, a blood pressure monitor, a pulse oximeter, or a heart rate monitor.

6. The non-transitory computer-readable media of claim 1, wherein the physiological data comprises hematological data, clinical biochemistry data, a glucose level of the user, a blood pressure of the user, an oxygen saturation level of the user, or a heart rate of the user.

7. The non-transitory computer-readable media of claim 1, wherein:
the first set of time slot data for the first task comprises a schedule for periodically performing the first task, and
the operations further comprise: modifying the schedule based on the first instance of physiological data.

8. The non-transitory computer-readable media of claim 1, wherein the operations further comprise:
using a machine learning algorithm to determine an additional task for the user based at least in part on the first instance of physiological data; and
adding, to the set of task records stored in the data repository, an additional task record corresponding to the additional task;
wherein the additional task record comprises (a) a second set of time slot data for the additional task indicating when the additional task is to be initiated, executed, and/or completed, and (b) a second set of status data for the first task indicating that the second task has not been completed.

9. A method comprising:
storing, in a data repository of an application platform, a set of task records corresponding to a set of tasks to be completed by a user of a software application hosted on the application platform, a first task record of the set of task records comprising (a) a first set of time slot data for a first task indicating when the first task is to be completed, and (b) a first set of status data for the first task indicating that the first task has not been completed;
displaying a subset of tasks, of the set of tasks, in a schedule within a user interface of the software application, the subset of tasks being ordered in the schedule based on time slot data corresponding respectively to the subset of tasks;
displaying status data for the subset of tasks, the status data comprising the first set of status data indicating that the first task has not been completed;
obtaining a first instance of physiological data of the user from a biomedical sensor of a health monitoring device,
determining that the first task is completed based on the first instance of physiological data obtained from the biomedical sensor;
responsive to the determination that the first task is completed, updating the first set of status data to indicate that the first task has been completed; and
subsequent to updating the first set of status data, concurrently displaying within the user interface of the software application: (a) the subset of tasks including the first task and (b) the first set of status data indicating that the first task has been completed, wherein the method is performed by at least one device including a hardware processor.

10. The method of claim 9, wherein:
a second task record of the set of task records comprises (a) a second set of time slot data for a second task indicating when the second task is to be completed, and (b) a second set of status data for the second task indicating that the second task has not been completed; and
the method further comprises:
obtaining a second instance of physiological data of the user from the biomedical sensor of the health monitoring device, the second instance of physiological data indicating that the user has not completed the second task;
determining that the user has not completed the second task based on the time slot data corresponding to the second task and the second instance of physiological data; and
responsive to the determination that the user has not completed the second task, displaying an alert on a computing device of the user, the alert comprising an identifier of the second task.

11. The method of claim 10, wherein:
the second task comprises taking a particular medication;
wherein the second instance of physiological data indicates that the user has not taken the particular medication.

12. The method of claim 9, wherein [insert element from claim 10 comprises:
the first task comprises taking a particular medication;
wherein taking the particular medication results in a physiological change for the user that is indicated by the first instance of physiological data.

13. The method of claim 9, wherein the health monitoring device comprises a glucose monitor, a blood pressure monitor, a pulse oximeter, or a heart rate monitor.

14. The method of claim 9, wherein the physiological data comprises hematological data, clinical biochemistry data, a glucose level of the user, a blood pressure of the user, an oxygen saturation level of the user, or a heart rate of the user.

15. The method of claim 9, wherein:
the first set of time slot data for the first task comprises a schedule for periodically performing the first task, and
the method further comprises: modifying the schedule based on the first instance of physiological data.

16. The method of claim 9, further comprising:
using a machine learning algorithm to determine an additional task for the user based at least in part on the first instance of physiological data; and
adding, to the set of task records stored in the data repository, an additional task record corresponding to the additional task;
wherein the additional task record comprises (a) a second set of time slot data for the additional task indicating when the additional task is to be initiated, executed, and/or completed, and (b) a second set of status data for the first task indicating that the second task has not been completed.

17. A system comprising:
one or more hardware processors;
one or more non-transitory computer-readable media; and
program instructions stored on the one or more non-transitory computer-readable media that, when executed by the one or more hardware processors, cause the system to perform operations comprising:
storing, in a data repository of an application platform, a set of task records corresponding to a set of tasks to be completed by a user of a software application hosted on the application platform, a first task record of the set of task records comprising (a) a first set of time slot data for a first task indicating when the first task is to be completed, and (b) a first set of status data for the first task indicating that the first task has not been completed;
displaying a subset of tasks, of the set of tasks, in a schedule within a user interface of the software application, the subset of tasks being ordered in the schedule based on time slot data corresponding respectively to the subset of tasks;
displaying status data for the subset of tasks, the status data comprising the first set of status data indicating that the first task has not been completed;
obtaining a first instance of physiological data of the user from a biomedical sensor of a health monitoring device,
determining that the first task is completed based on the first instance of physiological data obtained from the biomedical sensor;
responsive to the determination that the first task is completed, updating the first set of status data to indicate that the first task has been completed; and
subsequent to updating the first set of status data, concurrently displaying within the user interface of the software application: (a) the subset of tasks including the first task and (b) the first set of status data indicating that the first task has been completed.

18. The system of claim 17, wherein:
a second task record of the set of task records comprises (a) a second set of time slot data for a second task indicating when the second task is to be completed, and (b) a second set of status data for the second task indicating that the second task has not been completed; and
the operations further comprise:
obtaining a second instance of physiological data of the user from the biomedical sensor of the health monitoring device, the second instance of physiological data indicating that the user has not completed the second task;
determining that the user has not completed the second task based on the time slot data corresponding to the second task and the second instance of physiological data; and
responsive to the determination that the user has not completed the second task, displaying an alert on a computing device of the user, the alert comprising an identifier of the second task.

19. The system of claim 18, wherein:
the second task comprises taking a particular medication;
wherein the second instance of physiological data indicates that the user has not taken the particular medication.

20. The system of claim 19, wherein:
the first task comprises taking a particular medication, wherein taking the particular medication results in a physiological change for the user that is indicated by the first instance of physiological data;
the health monitoring device comprises a glucose monitor, a blood pressure monitor, a pulse oximeter, or a heart rate monitor;
the physiological data comprises hematological data, clinical biochemistry data, a glucose level of the user, a blood pressure of the user, an oxygen saturation level of the user, or a heart rate of the user; and
the operations further comprise:
using a machine learning algorithm to determine an additional task for the user based at least in part on the first instance of physiological data; and
adding, to the set of task records stored in the data repository, an additional task record corresponding to the additional task;
wherein the additional task record comprises (a) a second set of time slot data for the additional task indicating when the additional task is to be initiated, executed, and/or completed, and (b) a second set of status data for the first task indicating that the second task has not been completed.
